(19) United States
(12) Patent Application Publication
(10) Pub. No.: US 2022/0091053 A1
(43) Pub. Date: Mar. 24, 2022

(54) METHOD FOR PREDICTING SOURCE ROCK BY PALEOENVIRONMENT RESTORATION

(71) Applicant: CHENGDU UNIVERSITY OF TECHNOLOGY, Chengdu (CN)

(72) Inventors: Huaguo WEN, CHENGDU (CN); Fei HUO, CHENGDU (CN); Wenli XU, CHENGDU (CN); Litao LUO, CHENGDU (CN); Yunbo RUAN, CHENGDU (CN); Jiale CHEN, CHENGDU (CN)

(21) Appl. No.: 17/357,947

(22) Filed: Jun. 24, 2021

(30) Foreign Application Priority Data
Sep. 21, 2020 (CN) 202010996114.1

(51) Int. Cl. G01N 23/20; G01N 33/24; G16C 20/20

(52) U.S. Cl. CPC G01N 23/20; G01N 33/241; G01N 2223/616; G01N 2223/056; G16C 20/20

(57) ABSTRACT

A method for predicting a source rock by paleoenvironment restoration includes: (1) measuring a content of each mineral; (2) judging whether a sedimentary environment is a marine facies or a non-marine facies by utilizing element combination forms of Sr/Ba, B/Ga, Th/U, Fe/Mn and Sr/Ca; (3) judging a specific numerical value of a paleosalinity through a boron element and comparing the same with a current normal seawater value to deduce whether the current sedimentary environment is a saline water or non-saline water sedimentary environment; (4) judging an oxidation or reduction environment during sedimentation through element combination forms of (Cu+Mo)/Zn and V/(V+Ni); and (5) comprehensively analyzing the sedimentary environment, restoring a relationship between a palaeosedimentary environment and a source-reservoir configuration.

### CROSS REFERENCES TO RELATED APPLICATIONS

The present application claims foreign priority of Chinese Patent Application No. 202010996114.1, filed on Sep. 21, 2020 in the China National Intellectual Property Administration, the disclosures of all of which are hereby incorporated by reference.

### TECHNICAL FIELD

The present invention relates to the field of shale reserve prediction technologies, and more particularly, to a method for predicting a source rock by paleoenvironment restoration.

### BACKGROUND

A source rock is also called an oil source rock. French petroleum geologist Tissot (1978) et al. defined the source rock as “an organic-rich rock generating a large amount of oil and gas and discharging the oil and gas”. The source rock is a rock capable of producing or having produced movable hydrocarbons.

Paleoenvironment characteristic analysis is very important for analyzing a relationship between sedimentary environment evolution and organic-rich shale formation, and even determines whether there is an effective source rock capable of generating the oil and gas. Sedimentary environment judgment has always aimed at core observation and description of a coring well section, and practical investigation and observation of a field profile, and then the paleoenvironment is studied by means of sediment grain size analysis, sedimentary mineral analysis, paleontology analysis, and clay mineral analysis. Moreover, at present, the prediction of the source rock mainly focuses on the qualitative or semi-quantitative identification of a high-quality source rock under single factor control (for example, the paleoenvironment is quantitatively analyzed only by a single index of a macro element and a micro element), which often has limitations, and during geological historical evolution, an abnormal environment also limits information fed back by the elements. Therefore, an existing method for predicting a source rock often has a problem of a poor accuracy of prediction results.

### SUMMARY

Aiming at the above defects in the prior art, the present invention provides a method for predicting a source rock by paleoenvironment restoration, which can greatly improve the accuracy of predicting the source rock, especially the high-quality favorable area for source rock development and distribution.

In order to achieve the above objective, the technical solutions used in the present invention are as follows.

A method for predicting a source rock by paleoenvironment restoration includes the following steps of:

(1) measuring a content of each mineral by macro and micro element experiments;

(2) according to the measurement results, judging whether a sedimentary environment is a marine facies or a non-marine facies by utilizing element combination forms of Sr/Ba, B/Ga, Th/U, Fe/Mn and Sr/Ca;

(3) judging a specific numerical value of a paleosalinity through a boron element and comparing the same with a current normal seawater value to deduce whether the current sedimentary environment is a saline water or non-saline water sedimentary environment;

(4) judging an oxidation or reduction environment during sedimentation through element combination forms of (Cu+Mo)/Zn and V/(V+Ni); and

(5) comprehensively analyzing the sedimentary environment judged in the steps (2) to (4), recovering a relationship between a palaeosedimentary environment and a source-reservoir configuration, analyzing a shale development and distribution rule according to the relationship, pointing out a reservoir-forming favorable combination, finally performing source rock evaluation with reference to drilling, oil testing and logging data, and finally predicting a relatively high-quality favorable area for source rock development and distribution.

Preferably, in the step (1), the contents of the minerals are measured by an X-ray diffraction experiment on a whole rock.

Further, in the step (3), a process of judging the specific numerical value of the paleosalinity includes: obtaining a clay mineral composition by X-ray analysis, then testing micro elements B, Ba, and Sr of a sample and a K₂O content, and finally calculating the paleosalinity by Walker and Adamas empirical formulas.

Further, after calculating the paleosalinity, the paleosalinity is further proved by utilizing carbon and oxygen isotopes according to a carbonate paleosalinity restoration formula.

Further, in the step (4), before judging the oxidation or reduction environment during sedimentation, a rare earth element is standardized through the North American shale.

Further, before comprehensively analyzing the sedimentary environment, the judged sedimentary environment is further proved by the following formulas:

$$\begin{matrix}{V}_{s} = {V}_{o} \times \frac{{N}_{Co}}{{S}_{Co} - l \times {T}_{Co}} & ( 1 ) \\ t = {S}_{La} \text{/} {N}_{La} & ( 2 ) \\ h = C \text{/} {V}_{s}^{\frac{3}{2}} & ( 3 )\end{matrix}$$

Wherein Vₛ represents a sedimentation rate when the sample is sedimented, in a unit of m/Ma; Vₒ represents a sedimentation rate under a normal environment, wherein a sedimentation rate of lake-delta mudstone is 0.2×10³ m/Ma to 0.3×10³ m/Ma; N_{Co} represents a mean abundance of Co in a normal lake sediment, which is 20 μg/g; S_{Co} represents an abundance of Co in the sample, which is 4.68 μg/g; t represents an influence of a Co element inputted from a terrestrial source on the sample; S_{La} represents a mean abundance of La in the sample, in a unit of μg/g; N_{La} represents a mean abundance of La in a clastic rock from the terrestrial source, which is 38.99 μg/g; C is a constant, which is 3.05×10⁵ and obtained by measuring a modern ocean water depth and a sedimentation rate; and h represents a paleowater depth, in a unit of m.

Compared with the prior art, the present invention has the following beneficial effects.

(1) According to the present invention, guided by a sequence stratigraphy theory, based on systematic observation of rock debris and core as well as sample analysis, combined with geophysical data such as logging, and depended on joint analysis by various analysis means (including marine facies or non-marine facies judgment, saline water or non-saline water sedimentary environment judgment, and oxidation or reduction environment judgment), core calibration and logging are used to establish a division and identification standard for different types of shales, and define vertical and horizontal distribution characteristics of different types of shales, then, based on analysis of core facies and single well facies, a sedimentary environment of an organic-rich shale is pointed out through identification of a geochemical indicator of a micro element, a main controlling factor of organic matter enrichment is discussed, a sedimentary mode of the organic-rich shale is put forward, a shale development and distribution rule is analyzed, and finally a relatively high-quality favorable area for source rock development and distribution is predicted. According to the present invention, a plurality of analysis means and results are reasonably integrated, since some parameters may be affected to be abnormal during sedimentation, a reliability of palaeoenvironment restoration is greatly improved through joint analysis through a plurality of parameters, so that the accuracy of predicting the source rock, especially the high-quality favorable area for source rock development and distribution, is greatly improved, and a good pre-foundation is provided for subsequent research such as finding a genetic relationship between crude oil and the source rock.

(2) According to the present invention, all-round evidences are supplemented during analysis, such as an evidence of a paleosalinity calculation value; before judging the oxidation or reduction environment during sedimentation, the rare earth element is standardized through the North American shale first to provide an evidence for analysis of the oxidation or reduction environment during sedimentation; and the judged sedimentary environment is further proved before comprehensively analyzing the sedimentary environment. In this way, an accuracy and a reliability of prediction are further improved through combination of multi-angle analysis, evidences, and analysis results.

(3) The present invention is simple in process, convenient in calculation, and strong in scheme feasibility, and is very suitable for large-scale popularization and application in rock reserve prediction.

### BRIEF DESCRIPTION OF THE DRAWINGS

FIG. 1 is a flow chart of the present invention.

FIG. 2 is a schematic diagram of X-ray diffraction measurement results of mineral contents of samples in an embodiment of the present invention.

FIG. 3 is a reference diagram for judging whether a sedimentary environment of an exploratory well is a marine facies or a non-marine facies in the embodiment of the present invention, wherein FIG. 3a is a schematic diagram of analysis of Sr/Ba; FIG. 3b is an analysis diagram of B/Ga; FIG. 3c is an analysis diagram of Th/U; FIG. 3d is an analysis diagram of Fe/Mn; and FIG. 3e is an analysis diagram of Sr/Ca.

FIG. 4 is a reference diagram for analyzing a paleosalinity of the exploratory well by utilizing a boron element in the embodiment of the present invention.

FIG. 5 is a reference diagram for judging whether the sedimentary environment of the exploratory well is an oxidation or reduction environment in the embodiment of the present invention, wherein FIG. 5a is an analysis diagram of (Cu+Mo)/Zn; and FIG. 5b is an analysis diagram of V/(V+Ni).

### DETAILED DESCRIPTION

The present invention is further described hereinafter with reference to the accompanying drawings and the embodiment, and the manners of the present invention include but are not limited to the following embodiment.

### Embodiment

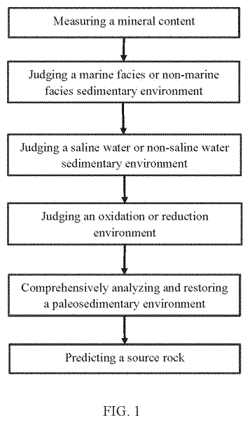

The present invention provides a method for predicting a source rock, which is implemented on the basis of paleoenvironmental restoration. Main flows include mineral content measurement, marine facies or non-marine facies sedimentary environment judgment, saline water or non-saline water sedimentary environment judgment, oxidation or reduction environment judgment, comprehensive analysis and restoration of paleoenvironment, and prediction of source rock, as shown in FIG. 1.

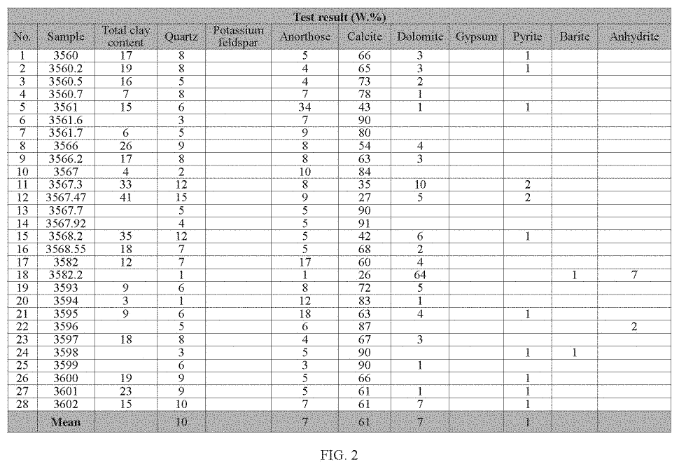

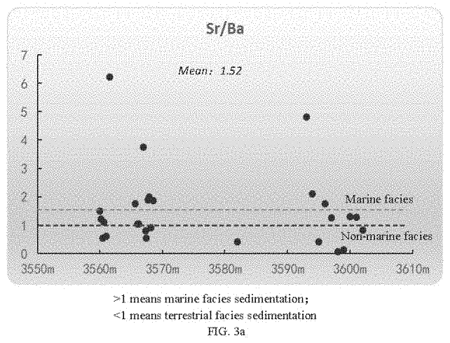

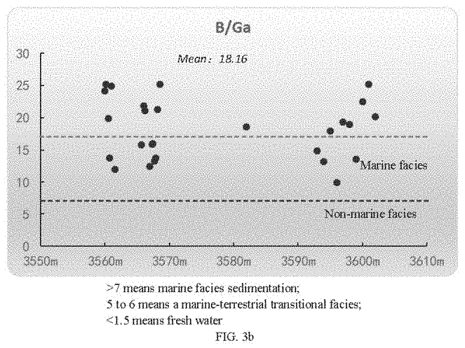

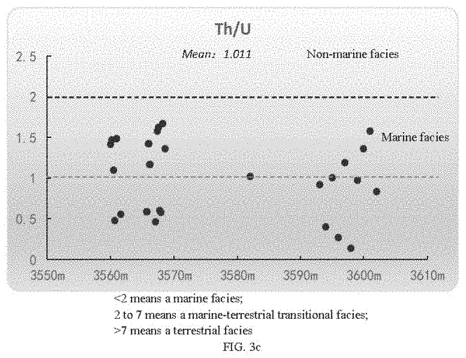

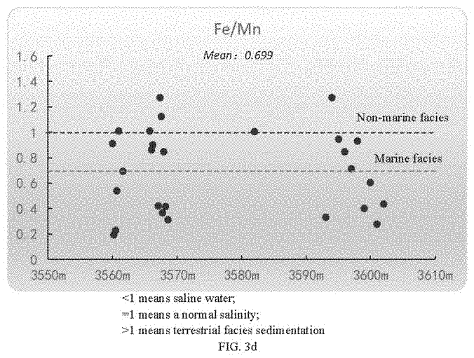

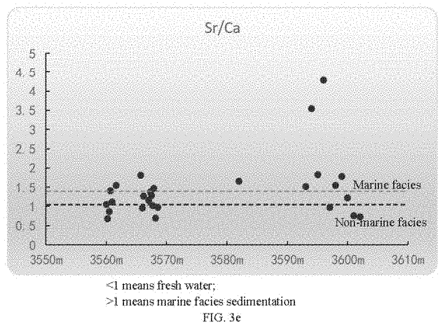

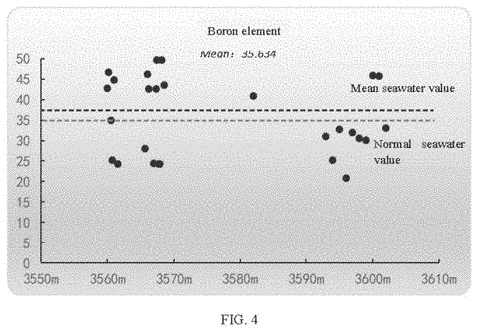

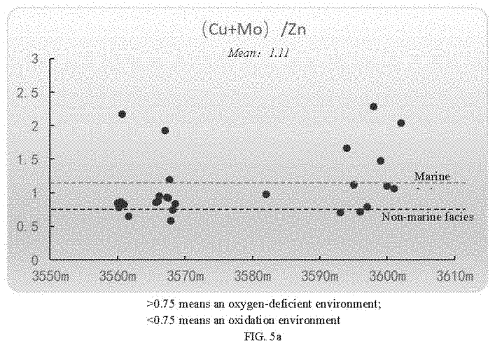

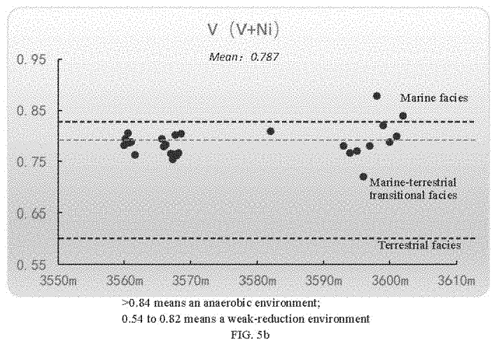

A content of each mineral is measured by macro and micro element experiments first (for example, samples are measured by an X-ray diffraction experiment on a whole rock, and results of the mineral content measurement are shown in FIG. 2). Then, according to the measurement results, whether a sedimentary environment is a marine facies or a non-marine facies is judged by utilizing element combination forms of Sr/Ba, B/Ga, Th/U, Fe/Mn and Sr/Ca. FIG. 3 shows reference for judging whether a sedimentary environment of an exploratory well is a marine-facies or a non-marine facies, and a judgment result of the exploratory well in a Leisan² period is a marine facies and saline water sedimentary environment (some samples may have a trend of brackish water, which may be due to mixing of terrigenous debris through analysis, and presence of a certain content of anorthose is also shown in the X diffraction of the whole rock).

Then, a specific numerical value of a paleosalinity is judged through a boron element and the specific numerical value is compared with a current normal seawater value to deduce whether the current sedimentary environment is a saline water or non-saline water sedimentary environment. In the embodiment, after obtaining a clay mineral composition by X-ray analysis, micro elements B, Ba, and Sr of a sample and a K₂O content are tested, and then the paleosalinity is calculated by Walker and Adamas empirical formulas.

Adamas empirical formula: Sp=0.0977X−7.043

Walker correction formula: “B”=8.5×B sample/K₂O sample.

FIG. 4 shows reference for analyzing a paleosalinity of the exploratory well by utilizing a boron element, a paleosalinity of the analyzed exploratory well in a Leisan² period is 36.634, which is the marine facies and saline water sedimentary environment, and is close to 35% of current normal seawater value.

After calculating the paleosalinity, the paleosalinity may be further proved by utilizing carbon and oxygen isotopes according to a carbonate paleosalinity restoration formula (put forward by Keith et al. in 1964).

A carbonate paleosalinity recovery formula is Z=2.048(¹³C+50)+0.498(¹⁸O+50).

Then, a rare earth element is standardized through the North American shale, and then an oxidation or reduction environment is judged during sedimentation through element combination forms of (Cu+Mo)/Zn and V/(V+Ni). FIG. 5 shows reference for judging an oxidation or reduction environment of the exploratory well, the sedimentary environment of the analyzed exploratory well in a Leisan² sub-section has a reduction-weak reduction environment. It is deduced that due to the mixing of terrigenous debris or a depth of water body lower than that of open sea, some data are not shown obviously, and located near a weak reduction bound. For example, some data of V/(V+Ni) are located near a weak reduction-reduction bound, which is a non-oxidization environment.

Then, the above judged sedimentary environment is comprehensively analyzed, a relationship between a palaeosedimentary environment and a source-reservoir configuration is restored, a shale development and distribution rule is analyzed according to the relationship, a reservoir-forming favorable combination is pointed out, source rock evaluation is finally performed with reference to drilling, oil testing and logging data, and a relatively high-quality favorable area for source rock development and distribution is finally predicted.

After predicting the relatively high-quality favorable area for source rock development and distribution, oil and gas sources may be compared, such as being compared by a fingerprint method, to find a genetic relationship between crude oil and the source rock.

The present invention is reasonable and rigorous in design, and the prediction results have strong reference. An analysis link and a proving link are both interlocked and complementary, thus making an important technical contribution to accurately predicting the high-quality favorable area for source rock development and distribution. That is to say, the present invention well breaks through limitations of the prior art, conforms to a trend of scientific and technological development, and well matches research needs of shale development and distribution rule at the current stage.

The above embodiment is only one of the preferred implementation of the present invention, and should not be used to limit the scope of protection of the present invention. However, the technical problems solved by any meaningless changes or embellishments made on the basis of the main design idea and spirit of the present invention are still consistent with those of the present invention and should be included in the scope of protection of the present invention.

What is claimed is:

1. A method for predicting a source rock by paleoenvironment restoration, comprising the following steps of:
(1) measuring a content of each mineral by macro and micro element experiments;
(2) according to the measurement results, judging whether a sedimentary environment is a marine facies or a non-marine facies by utilizing element combination forms of Sr/Ba, B/Ga, Th/U, Fe/Mn and Sr/Ca;
(3) judging a specific numerical value of a paleosalinity through a boron element and comparing the same with a current normal seawater value to deduce whether the current sedimentary environment is a saline water or non-saline water sedimentary environment;
(4) judging an oxidation or reduction environment during sedimentation through element combination forms of (Cu+Mo)/Zn and V/(V+Ni); and
(5) comprehensively analyzing the sedimentary environment judged in the steps (2) to (4), recovering a relationship between a palaeosedimentary environment and a source-reservoir configuration, analyzing a shale development and distribution rule according to the relationship, pointing out a reservoir-forming favorable combination, finally performing source rock evaluation with reference to drilling, oil testing and logging data, and finally predicting a relatively high-quality favorable area for source rock development and distribution.

2. The method for predicting the source rock by paleoenvironment restoration according to claim 1, wherein in the step (1), the contents of the minerals are measured by an X-ray diffraction experiment on a whole rock.

3. The method for predicting the source rock by paleoenvironment restoration according to claim 1, wherein in the step (3), a process of judging the specific numerical value of the paleosalinity comprises: obtaining a clay mineral composition by X-ray analysis, then testing micro elements B, Ba, and Sr of a sample and a K₂O content, and finally calculating the paleosalinity by Walker and Adamas empirical formulas.

4. The method for predicting the source rock by paleoenvironment restoration according to claim 3, wherein after calculating the paleosalinity, the paleosalinity is further proved by utilizing carbon and oxygen isotopes according to a carbonate paleosalinity restoration formula.

5. The method for predicting the source rock by paleoenvironment restoration according to claim 4, wherein in the step (4), before judging the oxidation or reduction environment during sedimentation, a rare earth element is standardized through the North American shale.

6. The method for predicting the source rock prediction by paleoenvironment restoration according to claim 1, wherein before comprehensively analyzing the sedimentary environment, the judged sedimentary environment is further proved by the following formulas:
$$\begin{matrix}{V}_{s} = {V}_{o} = \frac{{N}_{Co}}{{S}_{Co} - l \times {T}_{Co}} & ( 1 ) \\ t = {S}_{La} \text{/} {N}_{La} & ( 2 ) \\ h = C \text{/} {V}_{s}^{\frac{3}{2}} & ( 3 )\end{matrix}$$
wherein Vₛ represents a sedimentation rate when the sample is sedimented, in a unit of m/Ma; Vₒ represents a sedimentation rate under a normal environment, wherein a sedimentation rate of lake-delta mudstone is 0.2×10³ m/Ma to 0.3×10³ m/Ma; N_{Co} represents a mean abundance of Co in a normal lake sediment, which is 20 μg/g; S_{Co} represents an abundance of Co in the sample, which is 4.68 μg/g; t represents an influence of a Co element inputted from a terrestrial source on the sample; S_{La} represents a mean abundance of La in the sample, in a unit of μg/g; N_{La} represents a mean abundance of La in a clastic rock from the terrestrial source, which is 38.99 μg/g; C is a constant, which is 3.05×10⁵ and obtained by measuring a modern ocean water depth and a sedimentation rate; and h represents a paleowater depth, in a unit of m.
